(19) United States
(12) Patent Application Publication
(10) Pub. No.: US 2021/0128054 A1
(43) Pub. Date: May 6, 2021

(54) Apparatus and Method for Viability Assessment of Tissue

(71) Applicant: INTHESMART Co., Ltd., Seoul (KR)

(72) Inventors: Choonghee LEE, Seoul (KR); Ilhyung SHIN, Jeju (KR)

(73) Assignee: INTHESMART Co., Ltd., Seoul (KR)

(21) Appl. No.: 17/073,352

(22) Filed: Oct. 18, 2020

Related U.S. Application Data
(60) Provisional application No. 62/928,502, filed on Oct. 31, 2019.

(51) Int. Cl. A61B 5/00; A61B 5/026; G16H 10/60; G16H 50/30

(52) U.S. Cl. CPC A61B 5/4227; A61B 5/7264; A61B 2576/02; G16H 10/60; G16H 50/30; A61B 5/0261

(57) ABSTRACT

A system and a method for assessing a viability of the parathyroid gland, the system includes a memory unit for storing an image of a subject's parathyroid gland detected by a near-infrared sensor, an information extractor for extracting feature information from the image and a processor that includes a machine learning model into which the feature information is inputted, and generates a blood flow index of the parathyroid gland from the feature information based on the machine learning model, the method includes performing an image pickup of a subject's parathyroid region with a near-infrared sensor, extracting feature information from an image of the subject's parathyroid region, and generating a blood flow index of the parathyroid gland from the feature information based on a machine learning model.

### CROSS-REFERENCE TO PRIOR APPLICATIONS

This application claims the benefit of U.S. Provisional Patent Application No. 62/928,502, filed on Oct. 31, 2019, which is all hereby incorporated by reference in its entirety.

### TECHNICAL FIELD

The present invention relates to assessing a viability of tissue, more particularly, to apparatus and methods for assessing a viability of parathyroid glands during a surgery in the thyroid gland region.

### DESCRIPTION OF THE RELATED ART

The parathyroid gland is an endocrine organ attached to the thyroid gland, and is generally composed of four small tissues in the upper, lower, left and right areas of the thyroid gland. This parathyroid gland secretes parathormone and regulates the metabolism of calcium and phosphorus in body fluids. When the parathyroid gland is not normally active or is removed, calcium in the blood decreases and a specific muscle spasm occurs throughout all body parts.

When performing surgery on the parathyroid gland, it is necessary to accurately identify the position of the parathyroid gland. Conventionally, it was identifying the position of the parathyroid gland by irradiating light with a specific wavelength to the thyroid gland region after allowing a patient to take a contrast agent, but this has a problem that makes the patient feel a psychological burden.

In addition, in the surgical process, it may be necessary to determine whether or not to remove the parathyroid gland according to viability of the parathyroid gland. In such a case, assessing the viability of the parathyroid gland was entirely dependent on the empirical judgment of the operator. Therefore, different results may be derived according to the individual experience differences of the operator, and it may cause a problem that reliability is greatly degraded due to inappropriate judgments.

Particularly, accurate identification, viability assessment and careful preservation of tissue anatomy are critical for reducing complications and improving surgical outcomes. Human vision is limited in clearly discriminating these structures and status. Unintended and/or unrecognized injuries to tissue result in short- and long-term morbidity and avoidable mortality. Thus, in many clinical scenarios, where accurate identification of tissue type, and precise assessment of tissue perfusion/viability are critical, current standard of visual examination and palpation relying on individual surgeon's experience has limitations.

Also, surgical resection of diseased tissue is a common procedure in general surgery. Determining exact margins of resection is solely based on tissue viability and sufficient blood supply. For example, it is often difficult to decide resection margins of intestine where there is no clear demarcation with undefined viability. If the lesion is extensive and susceptible to short bowel syndrome, acute mesenteric ischemia, and necrotizing enterocolitis, surgeons tend to make hard surgical decisions. Inadequate bowel resection leads to sepsis from remained necrotic bowel, whereas massive bowel resection leads to short bowel syndrome. In case of insufficient blood supply, anastomotic leak and stricture can occur. Therefore, accurate intraoperative assessment of tissue viability is crucial. However, there are not standardized and no practical equipment readily available.

Therefore, a heretofore unaddressed need exists in the art to address the aforementioned deficiencies and inadequacies.

### SUMMARY OF DISCLOSURE

In one aspect, the present invention provides a system for assessing a viability of the parathyroid gland, the system includes a memory unit for storing an image of a subject's parathyroid gland detected by a near-infrared sensor; an information extractor for extracting feature information from the image; and a processor that includes a machine learning model into which the feature information is inputted, and generates a blood flow index of the parathyroid gland from the feature information based on the machine learning model.

In another aspect, the present invention provides a system for assessing a viability of the parathyroid gland, the system includes a memory unit for storing an image of a subject's parathyroid gland detected by a near-infrared sensor; an information extractor for extracting feature information from the image; and a processor that includes a look-up table in in which a blood flow index based on a reference feature information is stored in advance, and generate the blood flow index by comparing and matching the feature information of the image of the subject's parathyroid gland with the reference feature information.

In embodiments, the system may further include a light source unit that irradiates light of a selected wavelength among wavelength bands ranging from 780 nm to 840 nm to a parathyroid region of the subject.

In embodiments, the feature information may include at least one of a speckle contrast value (K) having information on blood flow, a distance (r) between a point where a near-infrared light is irradiated to the parathyroid region and the parathyroid region detected through the near-infrared sensor, and a time (T) at which the parathyroid region is exposed by the near-infrared light.

In embodiments, the feature information may include clinical information of the subject, the clinical information includes any one of the subject's age, sex, a medical history, exercise habits, eating habits, smoking and alcohol consumption.

In embodiments, the machine learning module may include at least one of a deep neural network (DNN), a convolutional neural network (CNN), and a recurrent neural network (RNN).

In further aspect, the present invention provides a method for assessing a viability of the parathyroid gland, the system includes performing an image pickup of a subject's parathyroid region with a near-infrared sensor; extracting feature information from an image of the subject's parathyroid region; and generating a blood flow index of the parathyroid gland from the feature information based on a machine learning model.

### BRIEF DESCRIPTION OF THE DRAWINGS

The patent or application file contains at least one drawing executed in color. Copies of this patent or patent application publication with color drawings will be provided by the Office upon request and payment of the necessary fee.

References will be made to embodiments of the invention, examples of which may be illustrated in the accompanying figures. These figures are intended to be illustrative, not limiting. Although the invention is generally described in the context of these embodiments, it should be understood that it is not intended to limit the scope of the invention to these particular embodiments.

FIG. 1 shows an exemplary thyroid gland and an exemplary parathyroid gland according to embodiments of the present invention.

FIG. 2 shows a schematic diagram of an apparatus for identifying a parathyroid gland and assessing a viability of the parathyroid gland according to embodiments of the present invention.

FIG. 3 shows a flow chart illustrating exemplary steps that identify the parathyroid gland and assess the viability of the parathyroid gland according to embodiments of the present invention.

FIG. 4 shows a schematic diagram of an apparatus that identifies a position of the parathyroid gland according to embodiments of the present invention.

FIG. 5 shows a view showing a color image and a first image acquired by an apparatus according to embodiments of the present invention.

FIG. 6 shows a schematic diagram of an apparatus that assesses a viability of the parathyroid gland according to embodiments of the present invention.

FIG. 7 shows a diagram illustrating a method of processing a second image acquired through an apparatus according to embodiments of the present invention.

FIGS. 8A and 8B are views showing a color image and a second image acquired by an apparatus according to embodiments of the present invention.

FIG. 9 shows a schematic diagram of an apparatus for assessing a viability of the parathyroid gland according to embodiments of the present invention.

FIG. 10 a schematic diagram that illustrates a first method for assessing a viability of the parathyroid gland using an apparatus according to embodiments of the present invention.

FIG. 11 a schematic diagram that illustrates a second method for assessing a viability of the parathyroid gland using an apparatus according to embodiments of the present invention.

### DETAILED DESCRIPTION OF THE PREFERRED EMBODIMENTS

In the following description, for purposes of explanation, specific details are set forth in order to provide an understanding of the invention. It will be apparent, however, to one skilled in the art that the invention can be practiced without these details. Furthermore, one skilled in the art will recognize that embodiments of the present invention, described below, may be implemented in a variety of ways, such as a process, an apparatus, a system, a device, or a method on a tangible computer-readable medium.

Components shown in diagrams are illustrative of exemplary embodiments of the invention and are meant to avoid obscuring the invention. It shall also be understood that throughout this discussion that components may be described as separate functional units, which may comprise sub-units, but those skilled in the art will recognize that various components, or portions thereof, may be divided into separate components or may be integrated together, including integrated within a single system or component. It should be noted that functions or operations discussed herein may be implemented as components that may be implemented in software, hardware, or a combination thereof.

It shall also be noted that the terms “coupled” “connected” or “communicatively coupled” shall be understood to include direct connections, indirect connections through one or more intermediary devices, and wireless connections.

Furthermore, one skilled in the art shall recognize: (1) that certain steps may optionally be performed; (2) that steps may not be limited to the specific order set forth herein; and (3) that certain steps may be performed in different orders, including being done contemporaneously.

Reference in the specification to “one embodiment,” “preferred embodiment,” “an embodiment,” or “embodiments” means that a particular feature, structure, characteristic, or function described in connection with the embodiment is included in at least one embodiment of the invention and may be in more than one embodiment. The appearances of the phrases “in one embodiment,” “in an embodiment,” or “in embodiments” in various places in the specification are not necessarily all referring to the same embodiment or embodiments.

In the following description, it shall also be noted that the terms “learning” shall be understood not to intend mental action such as human educational activity because of referring to performing machine learning by a processing module such as a processor, a CPU, an application processor, microcontroller, so on.

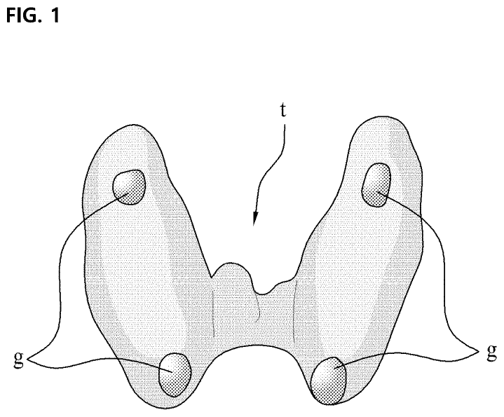

FIG. 1 shows an exemplary thyroid gland and an exemplary parathyroid gland according to embodiments of the present invention.

As depicted, in general, parathyroid glands (g) which is composed of four small tissues in the upper, lower, left and right areas of the thyroid gland are located behind the thyroid gland (t) located in the front center of the neck, and in general. As explained in the description of the related art, when performing surgery on the parathyroid gland (g), it is very important to accurately identify the position of the parathyroid gland (g) and to understand a viability of the parathyroid gland (g). Thus, according to an embodiment of the present invention, the apparatus and method that are capable of identifying the position of the parathyroid gland (g) using image information acquired from the thyroid gland using light having a wavelength of a specific region, and assessing the viability of the parathyroid gland (g) using the same are provided.

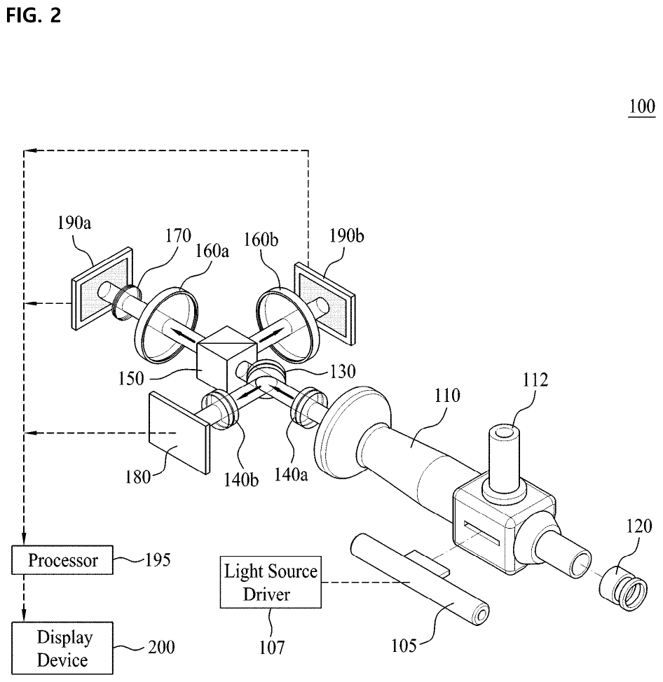

FIG. 2 shows a schematic diagram of an apparatus for identifying a parathyroid gland and assessing a viability of the parathyroid gland according to embodiments of the present invention.

As depicted, the apparatus 100 may include a light source unit 105, an endoscope assembly 110, a color sensor 180, a first near-infrared sensor 190a, a second near-infrared sensor 190b, and a light source driver 107. In the present document, identifying the parathyroid gland and assessing the viability of the parathyroid gland are described in conjunction with the thyroidectomy. However, it should be apparent to those ordinary skill in the art that the identifying and assessing may be performed as an intraoperative process during any other surgical procedures.

In embodiments, the light source unit 105 may be coupled to one side of the endoscope assembly 110 to irradiate light having a wavelength selected in a preset wavelength range to a parathyroid surgery region or the parathyroid gland. In embodiments, the light source unit 105 may irradiate light in a direction parallel to the light incident on the endoscope assembly 110 from the parathyroid surgery region. Although not shown in FIG. 2, the light source unit 105 can control an angle of the irradiated light in a predetermined range. It will be apparent to one skilled in the art that the light source unit 105 can be easily formed to control the angle of light irradiated.

In embodiments, the light source unit 105 may include a light emitting diode (LED) capable of generating light in a visible or near-infrared region, or a laser diode (LD) generating light in a near-infrared region. In this case, a wavelength of the near-infrared region can be selected from a wavelength band ranging from 780 nm to 840 nm.

In embodiments, when performing an image pickup by the color sensor 180, the first near-infrared sensor 190a and the second near-infrared sensor 190b, the light source unit 105 may irritate light to a corresponding region, e.g., parathyroid surgery region, parathyroid gland, for capturing an image. In alternative embodiments, the light source unit 105 may include a functional lens such as a diffusing lens, a focusing lens, so on to focus or diffuse light on the corresponding region.

In embodiments, the light source driving unit 107 may control the light source unit 105 and selectively controls a region of light generated from the light source unit 105. For example, the light source driving unit 107 can control a LED of the light source unit 105 irradiating visible light to be worked while the image pickup is performed by the color sensor 180, and control the LED of the light source unit 105 or the LD of the light source unit 105 irradiating near-infrared light to be operated while the image pickup is performed by the first near-infrared sensor 190a and the second near-infrared sensor 190b the near-infrared sensor 190b.

In embodiments, the endoscope assembly 110 is a medium for acquiring image information of a parathyroid surgery area to which light is irradiated from the light source unit 105. The endoscope assembly 110 may include a grip part 112 that enables a user to easily grip the endoscope assembly 110 and a polarizing cap 120 may be provided at a distal portion of the endoscope assembly 110. As it can be understood through the FIG. 2, since a detailed structure and operation process of the endoscope assembly 110 are apparent to those skilled in the art, a detailed description thereof will be omitted.

In embodiments, the color sensor 180 may implement a color image by detecting a visible region from the image information acquired through the endoscope assembly 110.

In embodiments, similarly, the first near-infrared sensor 190a may detects a first infrared region from image information acquired through the endoscope assembly 110 and implement a first image for identifying the position of the parathyroid gland, the second near-infrared sensor 190b may detect a second infrared region from image information acquired through the endoscope assembly 110 and implement a second image for assessing the viability of the parathyroid gland. In this case, the first infrared region and the second infrared region may have different wavelength bands. For instance, the first infrared region detected by the first near-infrared sensor 190a may be a wavelength band generated by irradiating light having a range of 780 nm to 805 nm from the light source unit 105 to the parathyroid gland, and the second infrared region detected by the second near-infrared sensor 190b may be a wavelength band generated by irradiating light having a range of 820 nm to 840 nm from the light source unit 105 to the parathyroid gland.

The apparatus 100 according to an embodiment of the present invention may further include a mirror 130 for reflecting visible light of the image information acquired through the endoscope assembly 110 toward the color sensor 180 and for transmitting infrared light of the image information toward the first near-infrared sensor 190a and the second near-infrared sensor 190b, a first lens 140a for passing through light of the image information before the light of image information reaches out to the mirror 130 and a second lens 140b for passing through visible light reflected by mirror 130.

In addition, the apparatus 100 according to an embodiment of the present invention may further include an infrared light splitter 150 for separating infrared lights of the first infrared region and the second infrared region and transmitting the infrared lights toward the first near-infrared sensor 190a and the second near-infrared sensor 190b, respectively, a first filter 160a for filtering infrared light of the first infrared region and a second filter 160b for filtering infrared light of the second infrared region.

It is noted that the apparatus 100 may include a polarizing lens 170 disposing between the first filter 160a and the first near-infrared sensor 190a. It is also noted that the apparatus 100 may further include a processor 195 for processing the color image, the first image, and the second image, and a display device 200 for displaying the first image and the second image processed by the processor 195.

In embodiments, the processor may be, but are not limited to a CPU or a memory for processing various images. In embodiments, it will be apparent to those of ordinary skilled in the art that the display device 200 can be used by applying any means such as an LCD capable of displaying the image.

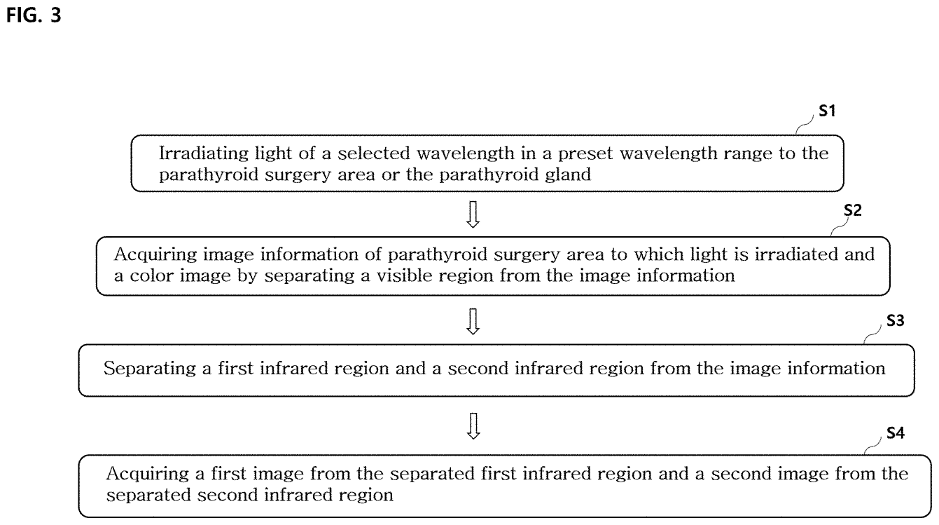

FIG. 3 shows a flow chart illustrating exemplary steps that identify the parathyroid gland and assess the viability of the parathyroid gland according to embodiments of the present invention.

As illustrated in the FIG. 3, the process starts at step S1. At step S1, the light source unit 105 irradiates light of a selected wavelength in a preset wavelength range to the parathyroid surgery area or the parathyroid gland. In this case, the selected wavelength may be a visible wavelength band or a near-infrared wavelength band.

Next, at step S2, the first near-infrared sensor 190a may acquires image information of a parathyroid surgery area to which light is irradiated and the color sensor 180 may acquires a color image by separating a visible region from the image information of the parathyroid surgery area.

At step S3, it may be separated by the first infrared region and the second infrared region from the image information. Then, at step S4, it may acquire a first image from the separated first infrared region to identify the position of the parathyroid gland and it may acquire a second image from the separated second infrared region to assess the viability of the parathyroid gland. In this case, acquiring the first image or acquiring the second image may be selectively performed.

Meanwhile, in alternative embodiments, the color image was obtained by irradiating light of the visible region and the near-infrared region. However, the color image may be acquired by irradiating only light of the visible region or by using natural light in a surgical environment without an operation of the light source unit. That is, it may acquire passively scene information from ambient lights without incitation light or any energy transfer. Thereafter, the first image and the second image may be obtained by irradiating light of the selected near-infrared region onto the parathyroid surgery region.

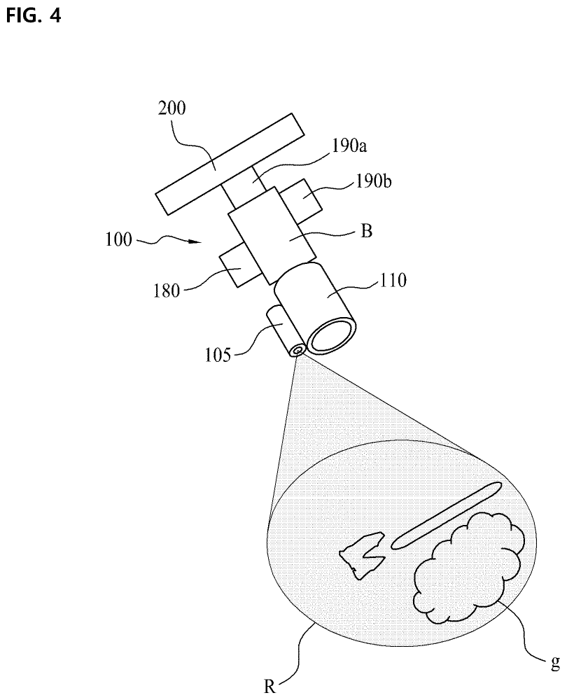

FIG. 4 shows a schematic diagram of an apparatus that identifies a position of the parathyroid gland according to embodiments of the present invention.

As depicted, on the process of identifying the position of the parathyroid gland, light is irradiated to the parathyroid surgery region R through the light source unit 105. In this time, the irradiated light may be diffused light, and may be near-infrared light of wavelength band ranging from 780 nm to 840 nm. It will be apparent to those of ordinary skill in the art that the diffused light may be easily generated by controlling any lens that is likely to be contained in the light source unit 105.

Thus, the reflected light generated from the parathyroid surgery region R is transmitted to the inside of the body (B) of the endoscope assembly 110, and the first image is implemented by the first near-infrared sensor 190a. At this time, in the first image, the parathyroid gland (g) located in the parathyroid gland surgery region (R) appears to have a higher luminous intensity than the other regions. Namely, the parathyroid gland g will emit autofluorescence in the first infrared region, which ranges from 780 nm to 805 nm. As such, the operator of the apparatus 100 can easily identify the position of the parathyroid gland g through the first image.

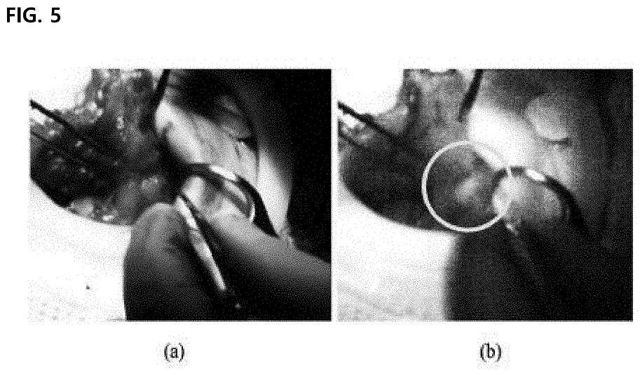

FIG. 5 shows a view showing a color image and a first image acquired by an apparatus according to embodiments of the present invention.

As depicted, on the first image (b), it is noted that a tissue shown in an area highlighted by yellow circle has a higher luminance than an ambient area by the autofluorescence thereof. Accordingly, the tissue may be identified by the parathyroid gland.

On the other hand, a position of the parathyroid gland can be identified as the autofluorescence of the parathyroid gland in the first infrared region, but a wrong position may be identified as the position of the parathyroid gland or other tissues may be confused by the parathyroid gland due to surface reflection generated in the other tissues of the thyroid region by the first infrared ray during determining the position of the parathyroid gland. To prevent an identification of the wrong position of the parathyroid gland, in embodiments of the present invention, a color image of the thyroid gland implemented using the color sensor 180 as shown in (a), and an autofluorescence image, e.g., the first image implemented from the parathyroid gland using the first near-infrared sensor 190a, the color image and the autofluorescence image may be overlayed each other by the processor 195, thereby generating a fusion image capable of mor visually distinguishing from other tissues. Then, the fusion image may be displayed on the display device 200. Thus, it is possible to increase the accuracy of identifying the location of the parathyroid gland.

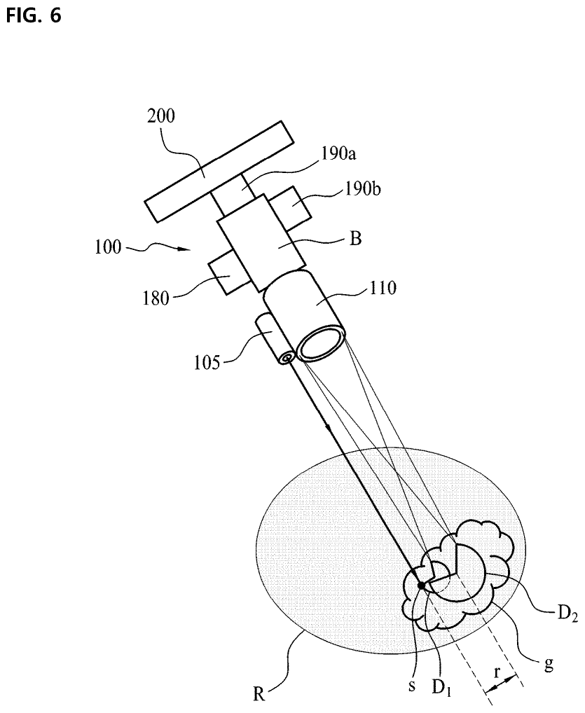

FIG. 6 shows a schematic diagram of an apparatus that assesses a viability of the parathyroid gland according to embodiments of the present invention.

As depicted, on the process of assessing the viability of the parathyroid gland, light is irradiated by focusing on a specific point (s) of the parathyroid gland (g) using the light source unit 105. In this time, the irradiated light may be light for focusing on the specific point (s), and may be near-infrared light of wavelength band ranging from 820 nm to 840 nm. It will be apparent to those of ordinary skill in the art that a focusing light may be easily generated by controlling any lens that is likely to be contained in the light source unit 105. Thus, diffuse speckle patterns (D1, D2) are generated from the parathyroid gland (g) by near-infrared light and the second image acquired by the second near-infrared sensor 190b may include speckle pattern information based on the diffuse speckle patterns. Thereafter, in embodiments, the apparatus 100 may assess the viability of the parathyroid gland using a diffuse speckle pattern information included into the second image.

In either case, although not depicted in the FIG. 6, on the process of assessing the viability of the parathyroid gland, light is irradiated by focusing on a region close to the parathyroid gland (g) using the light source unit 105. The irradiated light may be light for focusing on the region close to the parathyroid gland (g), and may be near-infrared light of wavelength band ranging from 820 nm to 840 nm, as described above. Thus, speckle patterns are diffused from a proximity region of the parathyroid gland (g) to the parathyroid gland (g), thereby generating the speckle patterns in the parathyroid gland (g). The speckle patterns may be converted into the second image including the speckle pattern information by the second near-infrared sensor 190b. Thereafter, similarly as described above, the apparatus 100 may assess the viability of the parathyroid gland using the diffuse speckle pattern information included into the second image.

Meanwhile, the diffuse speckle patterns may appear quantitatively or qualitatively differently depending on a distance (r) between a point at which the near-infrared light is irradiated and an area of the second image acquired by the second near-infrared sensor 190b. Thus, in embodiments, the apparatus 100 may optimize the distance (r) that can produce reliable results even if it performs quantitative or qualitative analysis on the diffuse speckle patterns.

In embodiments, a longitudinal axis of the light source unit 105 may be preferably disposed in a direction parallel to the longitudinal axis of the endoscope assembly 110. This is to prevent generation of noise due to near-infrared light when the speckle pattern information is obtained through the endoscope assembly 110 on the speckle patterns generated by the near-infrared light of the light source unit 105. That is, if a window view image acquired by the endoscope assembly 110 includes not only the region where the speckle patterns are generated but also the specific point (s) on where the near-infrared light is focused, the reliability of speckle pattern information due to the focusing light is degraded. Therefore, in embodiments, at least the area to which the focusing light of the light source unit is irradiated and the area of the window view image for obtaining the speckle pattern must be different from each other.

In addition, although not depicted in the Figures, in embodiments, the apparatus 100 may further include various sensor such as a temperature sensor for identifying the position of parathyroid gland and assessing the viability of parathyroid gland, the apparatus 100 may identify the position of parathyroid gland and assess the viability of the parathyroid gland using information obtained spatially or temporally from the sensors.

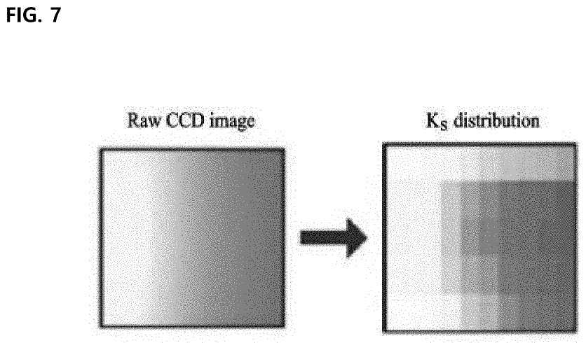

FIG. 7 shows a diagram illustrating a method of processing a second image acquired through an apparatus according to embodiments of the present invention.

As shown on the left side of FIG. 7, first, a raw image of the second image (Raw CCD image) may be obtained by the second near-infrared sensor 190b. After that, as shown of the right side of FIG. 7, speckle contrast value (Ks), which is information of a blood flow such as velocity of blood flow, may be calculated to a pixel of the raw image of the second image using a predetermined formula and a contrast map for the raw image is generated using the speckle contrast value (Ks). More detailed description of the predetermined formula is given below. Then, a color grayscale level of each pixel may be matched according to the contrast map to generate the second image.

In embodiments, the contrast map may be formed using at least one of temporal contrast, spatial contrast, and spatiotemporal contrast. For instance, in the temporal contrast case, the contrast map may be generated by calculating the speckle contrast values (Ks₁, Ks₂, Ks₃, for pixels of frame images constituting the raw image and comparing them with each other. In the spatial contrast case, the contrast map may be generated by dividing all pixels of the raw image into pixel groups, calculating the speckle contrast value (Ks₁) for one pixel included into each pixel group and the speckle contrast value (Ks₂) for the remaining pixels, and comparing them with each other. In the spatiotemporal contrast case, the contrast map may be generated by mixing the temporal contrast and the spatial contrast.

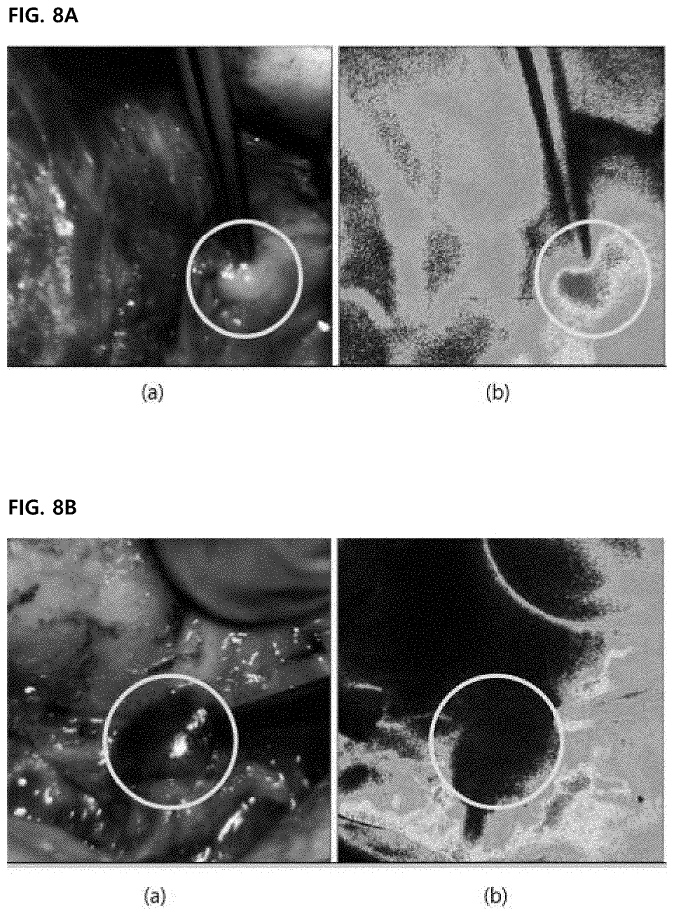

FIGS. 8A and 8B are views showing a color image and a second image acquired by an apparatus according to embodiments of the present invention.

As depicted in FIG. 8A, (a) is for the color image of the parathyroid gland acquired by the color sensor 180 and (b) is for the second image of the parathyroid gland acquired by the second near-infrared sensor 190b. The second image is an image obtained by corresponding a color grayscale with each pixel based on the contrast map which is described in FIG. 7 above. In the second image (b), it is shown that the parathyroid gland is biologically alive because the speckle contrast value (Ks) of pixels which correspond to the position of the parathyroid gland is less than a preset threshold value.

As depicted in FIG. 8B, (a) is for the color image of the parathyroid gland acquired by the color sensor 180 and (b) is for the second image of the parathyroid gland acquired by the second near-infrared sensor 190b. Similar to FIG. 8A, the second image is an image obtained by corresponding a color grayscale with each pixel based on the contrast map which is described in FIG. 7 above. In the second image (b), it is shown that the parathyroid gland is biologically dead because the speckle contrast value (Ks) of pixels which correspond to the position of the parathyroid gland exceeds a preset threshold value.

Meanwhile, under processing the second image, the speckle contrast value (K) may be derived as a one-dimensional numerical value through the following equations 1 to 4.

$${K}^{2} ( p , T ) = \frac{2 β}{T} {\int }_{0}^{T} {( 1 - τ / T ) [ {g}_{1} ( p , τ ) ]}^{2} dτ , \qquad [ Equation 1 ]$$

Here, K is a speckle contrast, T is exposure time that the parathyroid surgery area is exposed to the second near-infrared ray, g1 is an electric-field autocorrelation function, ρ is a distance between a light source and a detector, and τ is a delay time.

k_{D}(τ)=\sqrt{3μ′ₛμₐ+6μ′ₛ²k₀²αD_{B}τ}, [Equation 2]

Here, μ′s is a scattering coefficient, μa is an absorption coefficient, and αDB is a Blood flow index.

1/K²∝αDb (blood flow index) [Equation 3]

K=σ/I [Equation 4]

Here, σ is a standard deviation of speckle intensity, I is a mean intensity.

In Equation 4, the K value of the speckle contrast experimentally measured in a tissue, e.g., parathyroid gland is fitted to the K value of the speckle contrast in the theoretical model of Equation 3. Accordingly, the blood flow index (αDb) is derived by approximating the theoretical K value to the experimental K value.

Thus, the apparatus according to embodiments of the present invention may irradiate light having a selected first wavelength to the parathyroid surgery area, and accurately identify the position of the parathyroid gland through a light separation process after acquiring image information of the parathyroid surgery area, Also, the apparatus according to embodiments of the present invention may irradiate the light of a selected second wavelength to the parathyroid gland or an adjacent area of the parathyroid gland, may obtain a diffuse speckle pattern generated in the parathyroid gland, thereby performing viability assessment of the parathyroid gland with high reliability.

Meanwhile, the blood flow index (αDb) should be calculated inversely by obtaining the speckle contrast value (K) through an experiment, and then putting the speckle contrast value (K) into the non-linear equation 3. Therefore, a high computing power of the apparatus for assessing the viability of the parathyroid gland is required to solve by a mathematical inverse calculation method.

In embodiments, systems and methods capable of obtaining a blood flow index without complicated calculations using a machine learning model and assessing a viability of the parathyroid gland in real time may be provided.

The present methods and systems can be operational with numerous other general purpose or special purpose computing system environments or configurations. Examples of well-known computing systems, environments, and/or configurations that can be suitable for use with the system and method comprise, but are not limited to, personal computers, server computers, laptop devices, and multiprocessor Systems. Additional examples comprise set top boxes, programmable consumer electronics, network PCs, mini computers, mainframe computers, distributed computing environments that comprise any of the above systems or devices, and the like.

The processing of the disclosed methods and systems can be performed by software components. The disclosed system and method can be described in the general context of computer-executable instructions. Such as program modules, being executed by one or more computers or other devices. Generally, program modules comprise computer code, routines, programs, objects, components, data structures, etc. that perform particular tasks or implement particular abstract data types. The disclosed method can also be practiced in grid-based and distributed computing environments where tasks are performed by remote processing devices that are linked through a communications network. In a distributed computing environment, program modules can be located in both local and remote computer storage media including memory storage devices.

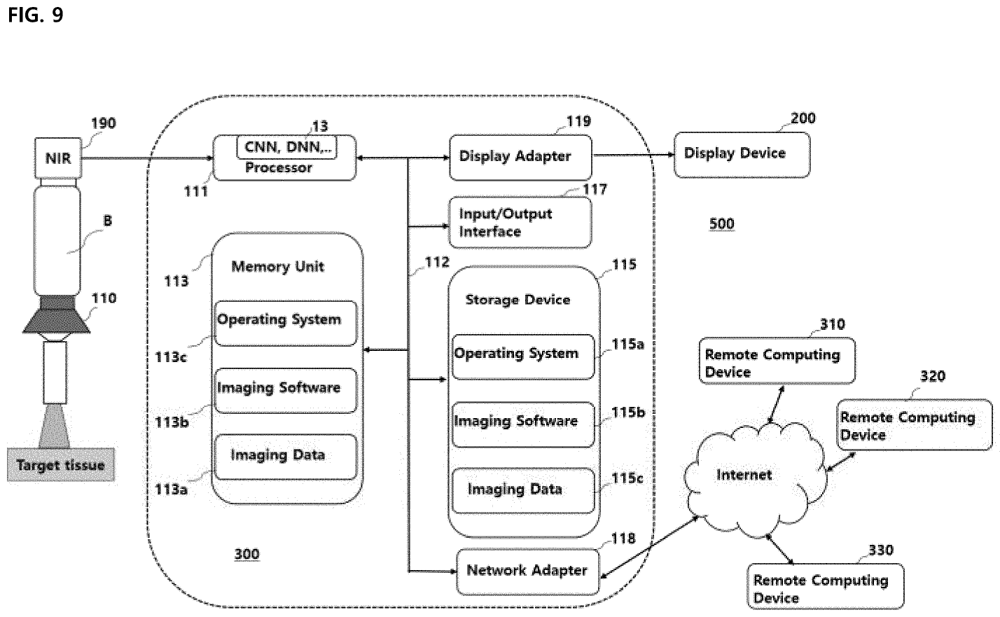

Further, one skilled in the art will appreciate that the systems and methods disclosed herein can be implemented via a computing device 300 shown in FIG. 9.

FIG. 9 shows a schematic diagram of a system for identifying assessing a viability of the parathyroid gland according to embodiments of the present invention.

As depicted, the system 500 may include an endoscope assembly 110, a near-infrared sensor 190, a computing device 300 having a processor 111 and a display device 200. The image of the parathyroid gland acquired through the endoscope assembly 110 may be photoelectrically converted into an image signal by the near-infrared sensor 190 and provided to the processor 111.

In embodiments, the computing device 300 may include, but are limited to, one or more processor 111 or processing units, a memory unit 113, a storage device 115, an input/output interface 117, a network adapter 118, a display adapter 119 and a system bus 112 connecting various system components including to the memory unit 113. The system 500 may further include the system bus 112 as well as other communication mechanism.

In embodiments, the processor 111 may be a processing module that automatically processes using a the machine learning model 13 and may be, but are limited to, a CPU (Computer Processing Unit), an AP (Application Processor), a microcontroller, a digital signal processor, so on. Also, the processor 111 may display an operation and a user interface of the system 500 on the display device 200 by communicating with a hardware controller for the display device 200 such a display adapter 119. The processor 111 may access the memory unit 113 and may execute commands stored in the memory unit 113 or one or more sequences of logic to control the operation of the system according to embodiments of the present invention to be described below. These commands may be read in the memory from computer readable media such as a static storage or a disk drive. In other embodiments, a hard-wired circuitry which is equipped with a hardware in combination with software commands may be used. The hard-wired circuitry can replace the soft commands. The logic may be an arbitrary medium for providing the commands to the processor 111 and may be loaded into the memory unit 113.

In embodiments, the system bus 112 may represent one or more of several possible types of bus structures, including a memory bus or memory controller, a peripheral bus, an accelerated graphics port, and a processor or local bus using any of a variety of bus architectures. By way of example, such architectures can comprise an Industry Standard Architecture (ISA) bus, a Micro Channel Architecture (MCA) bus, an Enhanced ISA (EISA) bus, a Video Electronics Standards Association (VESA) local bus, an Accelerated Graphics Port (AGP) bus, and a Peripheral Component Interconnects (PCI), a PCI-Express bus, a Personal Computer Memory Card Industry Association (PCMCIA), Universal Serial Bus (USB) and the like. Also, the system 112, and all buses specified in this description can also be implemented over a wired or wireless network connection and each of the Subsystems, including the processor 111, the memory unit 113, an operating system 113c, an imaging software 113b, an imaging data 113a, a network adapter 118, a storage device 115, an input/output interface 117, a display adapter 119 and a display device 200 may be contained within one or more remote computing devices 310,320,330 at physically separate locations, connected through buses of this form, in effect implementing a fully distributed system.

A transmission media including wires of the bus may include at least one of coaxial cables, copper wires, and optical fibers. For instance, the transmission media may take a form of sound waves or light waves generated during radio wave communication or infrared data communication.

In embodiments, the system 500 may transmit or receive the commands including messages, data, information, and one or more programs, i.e., an application code, through a network link or the network adapter 118.

In embodiments, the network adapter 118 may include a separate or integrated antenna for enabling transmission and reception through the network link. The network adapter 118 may access a network and communicate with a remote computing devices 310,320,330 such as a remote system for assessing a viability of the parathyroid gland.

In embodiments, the network may include at least one of LAN, WLAN, PSTN, and cellular phone networks, but is not limited thereto. The network adapter 118 may include at least one of a network interface and a mobile communication module for accessing the network. The mobile communication module may be accessed to a mobile communication network for each generation, e.g., 2G to 5G mobile communication network.

In embodiments, on receiving a program code, the program code may be executed by the processor 111 and may be stored in a disk drive of the memory unit 113 or in a non-volatile memory of a different type from the disk drive for executing the program code.

In embodiments, the computing device 300 may include a variety of computer readable media. Exemplary readable media can be any available media that is accessible by the computing device 300 and includes, for example, and not meant to be limiting, both volatile and non-volatile media, removable and non-removable media.

In embodiments, the memory unit 113 may store an operating system, a driver, an application program, data, and a database for operating the system 500 therein, but is not limited thereto. In addition, the memory unit 113 may include a computer readable medium in a form of a volatile memory such as a random access memory (RAM), a non-volatile memory such as a read only memory (ROM), and a flash memory. For instance, it may include, but is not limited to, a hard disk drive, a solid state drive, an optical disk drive, and the like.

In embodiments, each of the memory unit 113 and the storage device 115 may be program modules such as the imaging software 113b,115b and the operating systems 113c,115c that can be immediately accessed so that a data such as the imaging data 113a, 115a is operated by the processor 111.

In embodiments, the machine learning model 13 may be installed into at least one of the processor 111, the memory unit 113 and the storage device 115. The machine learning model 13 may include, but is not limited thereto, at least one of a deep neural network (DNN), a convolutional neural network (CNN) and a recurrent neural network (RNN), which are one of the machine learning algorithms.

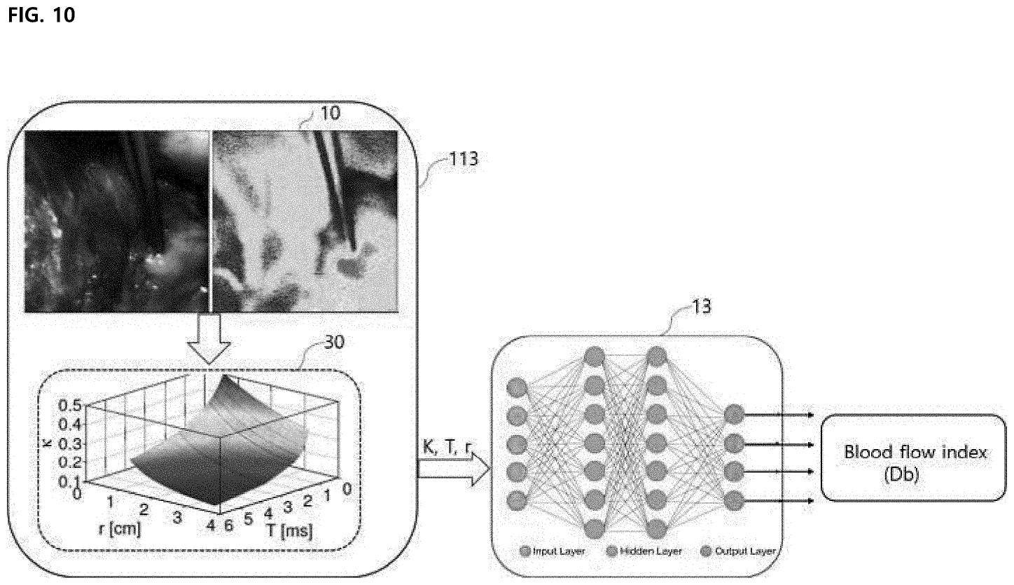

FIG. 10 shows a diagram illustrating a first method that assess a viability of a parathyroid gland through an system according to embodiments of the present invention.

As depicted, an image 10 of the parathyroid gland acquired by a near-infrared sensor (not shown) may be stored in the memory unit 113. The memory unit 113 may include an information extractor 30 and the information extractor 30 may extract feature information from the image of the parathyroid gland. In embodiments, the information extractor 300 is not included in the memory unit 113 and may be independently configured to be controlled by the processor. The feature information may include at least one of the speckle contrast value (K) having information on blood flow, a distance (r) described in FIG. 6, between a point where near-infrared light is irradiated to the parathyroid region in order to perform an image pickup of the parathyroid gland and the parathyroid region obtained through the near-infrared sensor, a time (T) at which the parathyroid region is exposed by the near-infrared light.

In addition, the feature information may further include clinical information of a subject who is a subject of the parathyroid gland. The clinical information may include any one of the subject's age, sex, a medical history, exercise habits, eating habits, smoking and alcohol consumption.

These feature information may be install to the machine learning model 13 stored in the processor 111 that is one of components for the system according to embodiments of the present invention. In the machine learning model 13, information on the blood flow of the parathyroid gland, i.e., a blood flow index (Db) is defined based on the feature information extracted from an image of the parathyroid gland for training previously obtained from a plurality of subjects. Since a method for generating the blood flow index is same with the method described above.

In embodiments, the system 500 may generate the blood flow index from the parathyroid gland image based on the machine learning model for the newly acquired parathyroid gland image having the feature information. Thus, it is possible to assess the viability of the parathyroid gland in real time based on the blood flow index (Db).

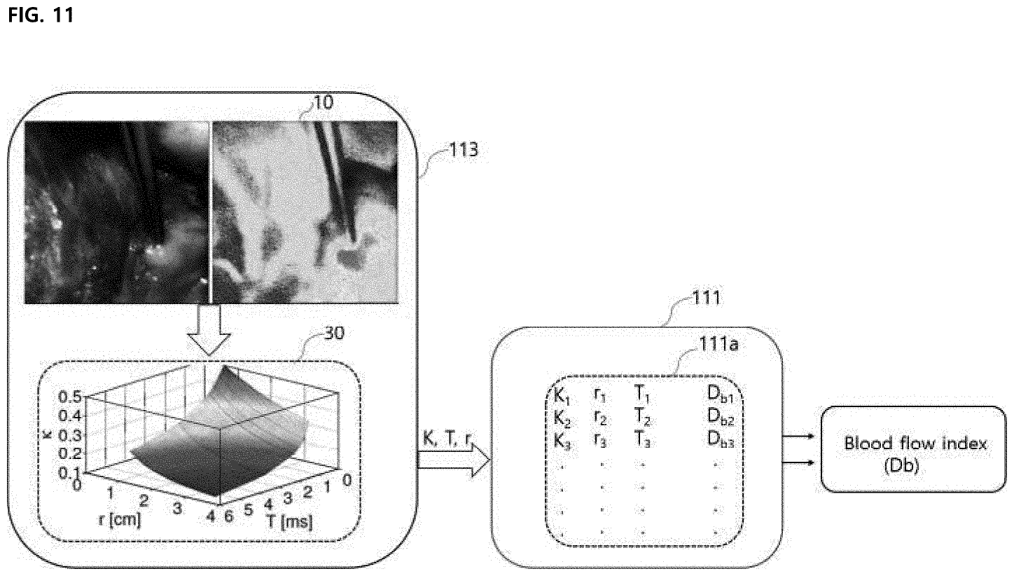

Meanwhile, in embodiments, the system 500 generates the blood flow index of the parathyroid gland using the machine learning model, but the system 500 may generate the blood flow index of the parathyroid gland using an algorithm or program with reference feature information set in advance. For instance, as depicted in FIG. 11, in other embodiments, a look-up table 111a in which the blood flow index based on the reference feature information is stored in advance may be included in the processor 111, and the processor 111 may generate the blood flow index by comparing and matching the feature information of the parathyroid gland image newly extracted by the information extractor 30 with the reference feature information. Thus, it is also possible to assess the viability of the parathyroid gland in real time based on the blood flow index (Db).

In such case, as described above, the image 10 of the parathyroid gland acquired by a near-infrared sensor (not shown) may be stored in the memory unit 113. The memory unit 113 may include an information extractor 30 and the information extractor 30 may extract feature information from the image of the parathyroid gland. In embodiments, the information extractor 300 may be included in the memory unit 113, but not be limited thereto. For instance, the information extractor 300 may be independently configured to be capable of controlling by the processor. In this case, the feature information may also include at least one of the speckle contrast value (K) having information on blood flow, a distance (r) described in FIG. 6, between a point where near-infrared light is irradiated to the parathyroid region in order to perform an image pickup of the parathyroid gland and the parathyroid region obtained through the near-infrared sensor, a time (T) at which the parathyroid region is exposed bt the near-infrared light.

It will be appreciated to those skilled in the art that the preceding examples and embodiment are exemplary and not limiting to the scope of the present invention. It is intended that all permutations, enhancements, equivalents, combinations, and improvements thereto that are apparent to those skilled in the art upon a reading of the specification and a study of the drawings are included within the true spirit and scope of the present invention.

What is claimed is:

1. A system for assessing a viability of the parathyroid gland, comprising:
a memory unit for storing an image of a subject's parathyroid gland detected by a near-infrared sensor;
an information extractor for extracting feature information from the image; and
a processor that includes a machine learning model into which the feature information is inputted, and generates a blood flow index of the parathyroid gland from the feature information based on the machine learning model.

2. The system of claim 1, further comprising, a light source unit that irradiates light of a selected wavelength among wavelength bands ranging from 780 nm to 840 nm to a parathyroid region of the subject.

3. The system of claim 1, wherein the feature information includes at least one of a speckle contrast value (K) having information on blood flow, a distance (r) between a point where a near-infrared light is irradiated to the parathyroid region and the parathyroid region detected through the near-infrared sensor, and a time (T) at which the parathyroid region is exposed by the near-infrared light.

4. The system of claim 1, wherein the feature information includes clinical information of the subject, the clinical information includes any one of the subject's age, sex, a medical history, exercise habits, eating habits, smoking and alcohol consumption.

5. The system of claim 1, wherein the machine learning module includes at least one of a deep neural network (DNN), a convolutional neural network (CNN) and a recurrent neural network (RNN).

6. A system for assessing a viability of the parathyroid gland, comprising:
a memory unit for storing an image of a subject's parathyroid gland detected by a near-infrared sensor;
an information extractor for extracting feature information from the image; and
a processor that includes a look-up table in in which a blood flow index based on a reference feature information is stored in advance, and generate the blood flow index by comparing and matching the feature information of the image of the subject's parathyroid gland with the reference feature information.

7. The system of claim 6, further comprising, a light source unit that irradiates light of a selected wavelength among wavelength bands ranging from 780 nm to 840 nm to a parathyroid region of the subject.

8. The system of claim 6, wherein the feature information includes at least one of a speckle contrast value (K) having information on blood flow, a distance (r) between a point where a near-infrared light is irradiated to the parathyroid region and the parathyroid region detected through the near-infrared sensor, and a time (T) at which the parathyroid region is exposed by the near-infrared light.

9. A method for assessing a viability of the parathyroid gland, comprising:
Performing an image pickup of a subject's parathyroid region with a near-infrared sensor;
Extracting feature information from an image of the subject's parathyroid region; and
Generating a blood flow index of the parathyroid gland from the feature information based on a machine learning model.

10. The method of claim 9, wherein the feature information includes at least one of a speckle contrast value (K) having information on blood flow, a distance (r) between a point where a near-infrared light is irradiated to the parathyroid region and the parathyroid region detected through the near-infrared sensor, and a time (T) at which the parathyroid region is exposed by the near-infrared light.

11. The method of claim 9, wherein the feature information includes clinical information of the subject, the clinical information includes any one of the subject's age, sex, a medical history, exercise habits, eating habits, smoking and alcohol consumption.

12. The method of claim 9, wherein the machine learning module includes at least one of a deep neural network (DNN), a convolutional neural network (CNN) and a recurrent neural network (RNN).
